(19) United States
(12) Patent Application Publication
(10) Pub. No.: US 2015/0291927 A1
(43) Pub. Date: Oct. 15, 2015

(54) SYSTEM SETUP FOR MONITORING AND/OR CONTROLLING FERMENTATION PROCESSES

(71) Applicant: BIOPROCESS CONTROL SWEDEN AB, Lund (SE)

(72) Inventor: Jing Liu, Lund (SE)

(21) Appl. No.: 14/439,811

(22) PCT Filed: Nov. 1, 2013
(86) PCT No.: PCT/SE2013/051285

§ 371 Date: Apr. 30, 2015

(30) Foreign Application Priority Data
Nov. 1, 2012 (SE) 1251237-2

(51) Int. Cl. C12M 1/36; C12P 3/00; C12Q 3/00

(52) U.S. Cl. CPC C12M 41/48; C12Q 3/00; C12P 3/00

(57) ABSTRACT

The present invention describes a system setup for monitoring and/or controlling one or multiple fermentation processes, said system setup comprising—at least one fermentation unit; a data acquisition unit; and a cloud computing unit having a database, a file storage capability, a data calculation capability and a user interface capability; wherein the at least one fermentation unit is connected to the acquisition unit which in turn is connected to the cloud computing unit so that on-line, real-time data on the one or multiple fermentation processes may be transferred from the at least one fermentation unit via the data acquisition unit to the cloud computing unit to be interpreted and displayed for a user being on-line, and wherein the system set-up enables measuring in the at least one fermentation unit and/or the data acquisition unit of the on-line, real-time data on the one or multiple fermentation processes, wherein the system setup also comprises—one or multiple laboratory simulation platform(s) and/or full-scale process(es) comprising said at least one fermentation unit, being in data connection with the cloud computing unit; and wherein the data acquisition unit is connected to the cloud computing unit so that all data acquisition, data interpretation and data storage is performed centralized on the cloud computing unit.

### FIELD OF THE INVENTION

The present invention relates to a system setup for monitoring and/or controlling one or multiple fermentation processes, especially for fermentation processes involving the production of gases, such as biogas or hydrogen.

### TECHNICAL BACKGROUND

The use of biogas as an energy carrier is one way to reduce the dependence on fossil-fuel based energy, thus exerting less impact on natural resources and on the environment. With the exponential increase of the applications of the anaerobic digestion processes for biodegradation of organic wastes and biogas production, there is an urgent need to increase knowledge and experience regarding such processes, improve the way of choosing the optimal substrate feeds, develop open and closed-loop control strategies, fine-tune start-up operations, identify stress factors and implement adjusted reactor designs. In fact, the knowledge and experience, to a large extent, will have a big impact on both the initial design, operational and economical details of a biogas plant.

The process of anaerobic degradation is highly complex and dynamic, where microbiological, biochemical and physico-chemical aspects are closely inter-related. For optimisation purposes, fermentation test methods at laboratory-scale are used to determine feedstock characteristics and simulate continuous operations of biogas reactors. In general, for broadly designed test programs, the combination of batch and continuous tests should be used. The continuous test simulates long-process conditions spanning a long time frame, whereas a large number of batch tests running in parallel deliver results on the influence of relevant parameter variations and feedstock characteristics. The objective of the fermentation tests in the defined continuous procedure is to obtain reliable long-term base data about the gas yield and its composition, and to build up as comprehensive a picture as possible regarding the degradation of the organic material, the course of the fermentation, and any problems in the degradation process which may occur. In fact, results from continuous lab-scale fermentation tests have on several occasions been proven to provide a good representation of the full-scale operation. With the help of continuous fermentation tests, it should also be possible to determine how the physico-chemical properties of the substrates affect the fermentation process and what process conditions must be put in place in order to achieve an optimal degradation and to maximise the gas yield. Continuous fermentation tests thus deliver the first useful information about the capabilities and the loading limits of the process, which is essential for designing and operating the biogas plant as well as for creating models concerning the economical feasibility of a project.

The large variety of biodegradation processes in the natural environment and in full-scale plants for treating wastewater and solid wastes has led to a rather large number of test methods based on different fermentation test procedures. Furthermore, fermentation tests in continuous procedures are laboratory-scale test methods subject to large variations, not only due to the heterogeneous nature of bio-wastes and bacteria culture used, but also due to the differences in the experiment setups and of the non-unified test protocols. For example, the reactor configuration, instrumentation and operational modes can all differ from one laboratory to another. In addition, the presentation of the results is not standardised either, which make comparability between two tests very difficult. Furthermore, the execution of a fermentation test in continuous mode is often a complex and very labour-intensive and time-consuming procedure, spanning a considerable period of time.

One aim of the present invention is to provide an improved system setup for monitoring and/or controlling continuous fermentation in terms of data acquisition, data interpretation and data storage.

### SUMMARY OF THE INVENTION

The stated purpose above is achieved by a system setup for monitoring and/or controlling one or multiple fermentation processes, said system setup comprising
at least one fermentation unit;
a data acquisition unit; and
a cloud computing unit having a database, a file storage capability, a data calculation capability and a user interface capability;
   wherein the at least one fermentation unit is connected to the acquisition unit which in turn is connected to the cloud computing unit so that on-line, real-time data on the one or multiple fermentation processes may be transferred from the at least one fermentation unit via the data acquisition unit to the cloud computing unit to be interpreted and displayed for a user being on-line, and wherein the system set-up enables measuring in the at least one fermentation unit and/or the data acquisition unit of the on-line, real-time data on the one or multiple fermentation processes,
   wherein the system setup also comprises
one or multiple laboratory simulation platform(s) and/or full-scale process(es) comprising said at least one fermentation unit, being in data connection with the cloud computing unit;
   and wherein the data acquisition unit is connected to the cloud computing unit so that all data acquisition, data interpretation and data storage is performed centralized on the cloud computing unit.

Before discussing the present invention in more detail, some definitions above may be further explained and discussed.

A “fermentation unit” may e.g. constitute a bioreactor according to the present invention, as is shown in FIG. 1.

“Cloud computing” represents the use of network based computing resources where the computation time and power comes from an external source. The word “cloud” refers to the specific hardware and software used. This means that the users do not necessarily need their own sets of hardware, saving thus both time and money. Furthermore, the expression “cloud computing unit” may also be seen as a “remote server” in terms of capability and means of usage.

The “database” is the intermediator between the hardware and the website. Here, raw sample data, event data, log data and instrument data (different states, when the instrument was last connected, etc) are stored. Using standard software allows the database to easily be migrated to another location if needed.

Moreover, a key feature of the present invention is the centralized mode of data acquisition, data interpretation and data storage. This has several advantages and is further discussed below. Furthermore, this is also an important difference when being compared to the known system used today. See for instance the systems disclosed in Gao L. et al., “Development of remote controlled lab scale bioreactor using virtual instrument technology”, ITME 2011-Proceedings: 2011 IEEE International Symposium on IT in Medicine and Education, 20111209-20111211, vol. 1, pages 15-17, and in Jagadeesh Chandra A. P et al., “Web-based collaborative 1-14 learning architecture for remote experiment on control of bioreactor's environment”, Journal of Software, April 2009, vol. 4, no. 2, pages 116-123 which are further disclosed below, where such data handling is performed locally and not centralized.

Some of the advantages with a system setup according to the present invention are:
High measurement performance (i.e. precision and accuracy) in continuous monitoring of key process parameters, e.g. biogas flow;
Long-term stability and large capacity for data logging and handling;
Standardisation of registered parameters, data interpretation and presentation to ensure reliable comparison of the results obtained at different laboratories and full-scale facilities;
User friendly interface for both easy experiment setup and operation follow-up, as well as possibility for online result visualisation;
High accessibility for process monitoring and control; and
Minimised time and labour demand, for both industrial and academic applications.

The use of software has been discussed before in the technical area of fermentation process, however not implemented in a setup mode as proposed according to the present invention. Moreover, the advantages disclosed above are not provided in any known existing solution today.

As an example relating to the use of software linked to fermentation processes, in WO2010/120230 there is discussed an in-house developed software program which is used together with a measuring device system in order to record, display and calculate data, as well as analyze the result. This is showed as a DAQ (data acquisition), which may be a computer bases recording made continuously and in real-time. Moreover, in WO2012/005667 there is also discussed online registration of gas flow on a computer unit by means of a gas flow meter. Not only the gas flow meter may be connected to the computer unit, but also other sensors/probes/detectors may be connected to the computer unit for online registration, such as detectors for pH, temperature, pressure, gas composition, slurry level etc.

None of WO2010/120230 or WO2012/005667 discloses a system setup according to the present invention, where the system comprises one or more fermentation unit(s); a data acquisition unit; and a cloud computing unit having a database, a file storage capability, a data calculation capability and a user interface capability. This is not disclosed or hinted in WO2010/120230 or WO2012/005667.

Moreover, remote control of fermentation and biogas production processes are also known. For example, in Gao L. et al., “Development of remote controlled lab scale bioreactor using virtual instrument technology”, ITME 2011-Proceedings: 2011 IEEE International Symposium on IT in Medicine and Education, 20111209-20111211, vol. 1, pages 15-17, there is disclosed a remote control configuration for a bioreactor system. The system may as such be operated over the internet. As such, the configuration comprises software consisting of five parts, namely signal acquisition and hardware control, on-line data storage and dynamic graphics, mathematical model and model based control, remote control, and the user interface parts. The program LabVIEW is used according to the article.

Furthermore, in Jagadeesh Chandra A. P et al., “Web-based collaborative 1-14 learning architecture for remote experiment on control of bioreactor's environment”, Journal of Software, April 2009, vol. 4, no. 2, pages 116-123, there is also shown a system corresponding to the one disclosed in the article according to Gao L. et al. where LabVIEW also is used.

As said above, none of the disclosed systems involve a centralized manner for data acquisition, data interpretation and data storage. This is a key feature according to the present invention.

### BRIEF DESCRIPTION OF THE DRAWINGS

FIG. 1 shows one possible embodiment of a system setup according to the present invention.

FIGS. 2-4 relate to results of an application test.

### SPECIFIC EMBODIMENTS OF THE INVENTION

Below the present invention, aspects thereof and different specific embodiments of the invention are further explained.

Cloud computing is today regarded as one of the most promising technologies to handle the high demands of tomorrow's information intense society. Some benefits with using the cloud are summarised below:
Flexibility/Elasticity. Users can freely choose to allocate resources after demand. This means that the user does not have to adjust the available computing power to the peak demand and thus buy and maintain a lot of unnecessary hardware. Today the average exploitation of data centres capacities is estimated to be 5-20% which means that there is an enormous surplus.
Accessibility. The cloud can easily be accessed from most types of platforms with an internet connection.
Reliability. As the cloud providers tend to distribute their data centres at multiple sites, there is a high likelihood that the resources from a failed one can be allocated to another one.
Sustainability. An improved and centralised resource utilisation will lead to less demand for energy and commodities.
Cost effective. Large scale and possibility to choose location (proximity to power plant, cold climate for cooling, etc.) gives and cheaper end product for the user.

With the continuous increase of scientific research activities today, there is a growing gap between the local storing and analysing capacity at the scientist's disposal and the amount of data generated. To solve this, cloud computing has been introduced as a solution that would improve the managing, analysing, storing and sharing of scientific data. Examples of these scientific cloud platforms are Open Science Data Cloud (OSDC) and Open Science Grid (OSG).

As mentioned above, the system set up according to the present invention exhibits several advantages. As mentioned, the centralized mode of data handling is a key feature. However, there are also other possible advantages according to specific embodiments of the present invention. According to one embodiment, all data acquisition, data interpretation and data storage being performed centralized is performed in a standardized manner. A standardized manner according to the present invention may encompass several different features and properties. A first aspect relates to data interpretation. In order to allow the data comparison and information sharing, all process parameters and data may need to be calculated in a way that can be well accepted by users. Therefore, certain well-defined standards should be followed for data presentation. One example is gas volume and flow rate. For example the gas volume and flow may be normalized at 0° C., 1 atm and moisture free condition. This will allow gas volume and flow rate to be compared from different data sources. A second aspect may relate to data presentation and storage. In order to allow the data comparison and information sharing, the data is presented and stored in the same way in terms of unit, time interval, resolution, etc. As hinted above, according to one embodiment of the present invention, the standardized manner implies that established well-defined standards and protocols are used for relevant data and data presentation. Moreover, according to yet another embodiment, the standardized manner implies the use of a pre-defined format for data storage and data presentation.

In line with the above disclosed, a third aspect is related to the sharing of data and information. Only when the data is collected in a central location, interpreted and presented in a standard manner, one is able to share and compare process performance. Therefore, according to one specific embodiment, the standardized manner implies data interpretation that allows for comparison and information sharing of the relevant data among users within a defined user group/community.

Moreover, it is preferable that the system set-up both involves a centralized and standardized manner according to above. The system configuration and design of the system according to the present invention can not only create fundamental base for gathering information from all users but also ensure the standardization in order to support the data statistic and comparison analysis. For example traditional SCADA system and in-house developed control system will never be able to meet the same or similar level of data sharing due to lack of centralized data storage and standardization, and hence will not provide the same level of value to all users or user community. Moreover, and as mentioned, since data acquisition, data interpretation, data presentation, reporting and storage are centralized with independent computing capacity data visualization can be done via various kind of platform from PC to tablet or smart phones. This is a very open feature with high remote accessibility and is very different in comparison to traditional SCADA system or in-house developed control system, where individual user can only access to the system information via defined platform, mainly PC or industrial PC and remote access is often limited to one system-to-one user or one system-to-limit user base. Furthermore, also the possibilities of forming user groups/communities and provide the possibility of common centralized and standardized way of sharing, storing and presenting data is also an important feature according to the present invention, and which is different from a traditional SCADA system.

According to yet another embodiment of the present invention, the system set-up comprises one or multiple laboratory simulation platform(s). As mentioned above, the present invention encompasses both the alternative with one or multiple laboratory simulation platform(s) or a full-scale process, or combinations thereof. In both of the cases, the process(es), i.e. the simulation process(es) or full-scale process(es), is(are) linked to the cloud computing unit and all data input is uploaded from the process in itself.

The present invention finds use in many different applications. According to one specific embodiment, the one or multiple fermentation processes are anaerobic or aerobic fermentation processes. One example of anaerobic such processes is anaerobic treatment of bio-wastes, e.g. anaerobic digestion for biogas production. An aerobic process may for instance be such treatment of wastewater.

According to one specific embodiment, the one or multiple fermentation processes are one or multiple biogas producing fermentation processes. As mentioned above, the fermentation unit(s) may be bioreactors. It should be mentioned that any type of reactor can be connected to the system setup according to the present invention through a gas inlet port. However, designing a gas tight reactor that allows liquid and gas mass transfer as well as continuous mixing and heating is a difficult task and many times systems like these are considered to be unreliable. Therefore, the system according to the present invention may include a verified set of reactors with all possible reactor configurations, such as CSTR (Continuous Stirred Tank Reactor), UASB (Upflow Anaerobic Sludge Blanket), EGSB (Expanded Granular Sludge Bed), IC (Internal Circulation), Biofilm, etc.

Furthermore, the system setup according to the present invention offers several unique functionalities that are important for the anaerobic fermentation tests in continuous procedures. One of them is automatic real-time gas normalisation. The system may automatically normalise the reported gas flow with regard to temperature, pressure and water vapour content. This is important since biogas is a compressible medium and the volume will thus be highly dependent of the gas pressure and temperature. Furthermore, biogas produced by anaerobic digestion is assumed to be saturated with water vapour and, in order to give accurate and precise quantitative gas measurements, the effect of the water should be minimised. Other unique features and advantages with the system setup according to the present invention are further disclosed below.

As understood from above, according to one specific embodiment of the present invention, the system set-up also includes one or several measuring devices and/or sensors. According to yet another embodiment, one or several measuring devices and/or sensors are positioned in the data acquisition unit. This should be seen as an alternative, and it should be noted that the measuring devices and/or sensors may be positioned differently in the system setup according to the present invention.

According to yet another embodiment of the present invention, the system set-up includes at least one gas flow measuring device. Moreover, the system set-up may also include specific sensor(s) measuring pH, temperature, pressure, gas composition, ORP (oxygen redox potential), alkalinity, (dissolved) hydrogen or (dissolved) oxygen, VFA (volatile fatty acid), biodegradable organic matter, or any fermentation metabolites as key process parameters. Combinations of different sensors are fully possible according to the invention. The sensors of interest and the combination of different such depend on the intended application.

According to one specific embodiment of the present invention, the system set-up also includes sensor(s) measuring pH, gas composition, (dissolved) hydrogen, or temperature, or a combination thereof. This is special interest as a minimum of sensors for monitoring and/or controlling fermentation processes where biogas is produced. Alternatives in relation to the feature of possible measuring devices and sensors are further discussed below, inter alia in the example.

Another unique advantage of the present invention is the possibility of automatic real-time calculation and visualisation of key process parameters. In order to have a common platform to compare process data and operation, there are a number of parameters that are used to standardise the process data. In the system of the present invention, both the organic loading rate (OLR) and the hydraulic retention time (HRT) may be calculated and presented in real-time together with the normalised gas flow. To reduce the data processing and transfer these graphs may be presented as running windows where the user can choose to display the data with various time intervals for detailed studies or with a longer time interval for more overall studies of the process. In such a report, other key process parameters such as specific gas production (SGP) and gas yield for both the total and organic input may be calculated and stored.

Yet another feature of interest is interactive feeding and discharging support. In order to provide a user friendly interface for experiment follow up, the system according to the present invention may include an advanced interactive system for substrate feeding and discharging of digested slurry. This may increase the process controlling capability of the system setup. The user then has the possibility to specify type of feedstock, its concentration, and schedule the time for feeding and one of the following variable inputs:

amount (weight), sought OLR or HRT. Depending on which parameter is chosen, the other two may be directly calculated to give the user the optimal support for how to feed and discharge the reactor.

Furthermore, the system setup may provide possibility to run in manual and automatic mode. For such automatic mode, no manual input for the feeding is necessary; instead the user should only specify the expected feeding volume and interval at the beginning of the process. This setting can also be changed at any time during the experiment. All the calculated process parameters are adjusted according to which mode is active.

Other possibilities that may be provided within the system setup are capability to generate standardised report with all recorded and interpreted data, such as data adjusted to a user defined sampling time interval (i.e. day, hour or quarter of hour), e.g. using linear piecewise interpolation, for easier visualisation, statistical analysis and modelling, and e.g. distant monitoring of experiments. Since all functions of the system in that regard are based in the cloud, the user will be able to follow the experiment regardless of geographic location, provided there is an internet connection and a compatible hardware platform (i.e. computer, smartphone or tablet). This also means that sharing experiment data and follow-up from different locations is extremely easy.

As explained above, the data acquisition unit or instrument may contain a measuring device or system, such as a flow cell array for ultra low gas flow, such as e.g. the flow cell device disclosed in WO10120229, which is used to detect the production of e.g. biogas or hydrogen depending on the application. This may e.g. be performed by water displacement and buoyancy, but also other alternatives are fully possible. Apart from the possible flow cell array, the instrument may contain the majority of the electronic parts, including an embedded controller running proprietary software on top of an operating system, such as Linux. The embedded controller handles all the registration of flow cell openings, maintains a connection to the database and uploads all the system generated data (i.e. time, pressure, temperature for flow cell openings) in real-time as soon as the event occurs. In case of a broken connection, the system retains data that has not been properly uploaded and sends them again automatically as soon as the connection is restored. Ample storage has been set aside for this caching so even extremely unstable internet connections can be used in a safe way.

Each system according to the present invention may be assigned to a specific user account and both control of the system and uploading of data from the system may be connected to this account. As such, the system may be designed so that there is no way for a system to access data generated by another system or for a not assigned user to control the system. As hinted above, according to one specific embodiment of the present invention the data acquisition unit holds both on-line, real-time data on the one or multiple fermentation processes and also user identity information to transfer data to a correct user account in the cloud computing unit.

The system may support DHCP (Dynamic Host Configuration Protocol) out of the box and automatically connect to the database upon connection so if the user's network supports it, the solution is completely plug-and-play with no needed user intervention. Should more specific configurations be needed, the system may support this as well.

According to the present invention there is also provided the use of a system setup. According to one specific embodiment there is provided the use of a system according to the present invention, for monitoring and/or controlling one or multiple fermentation processes. According to one specific embodiment, said one or multiple fermentation processes are biogas producing fermentation processes. According to yet another specific embodiment of the present invention, the use includes measuring at least one biogas flow which is the on-line, real-time data on the one or multiple fermentation processes.

According to yet another specific embodiment, the at least one biogas flow is compensated with reference to temperature and pressure, which parameters are also measured. Moreover, one further embodiment includes the use of a system setup according to the present invention, and where monitoring and/or controlling is performed continuously on one or multiple continuous fermentation processes.

### DETAILED DESCRIPTION OF THE DRAWINGS AND AN EXAMPLE

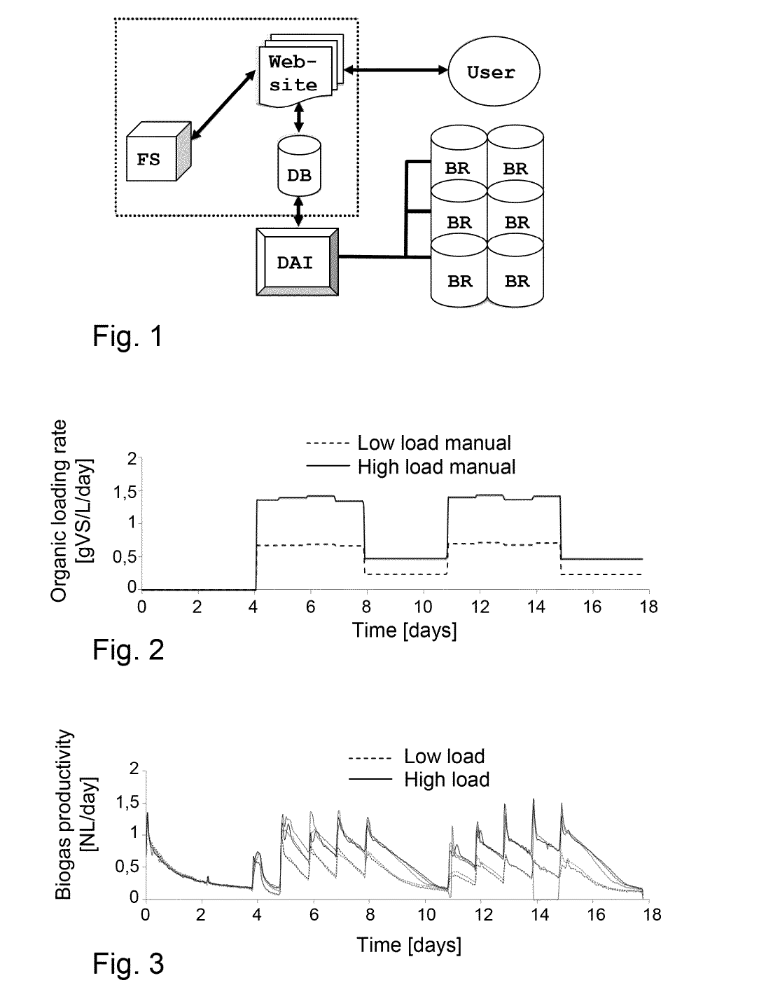

In FIG. 1 there is shown one example of a system setup according to the present invention. The system setup according to the specific embodiment comprises five main parts, namely bioreactor(s) (BR), data acquisition instrument (DAI), the website, the database (DB) and the file storage (FS). All these parts work together to provide the functionality required of the system to work as quickly, reliably and securely as possible. The website, database and file storage together form the cloud part of the system, marked with dots. However, it is important to understand that this is just an example, and fact is that for instance the cloud computing unit may have a different design, for instance where the file storage capability is part of the data base, which inter alia is a server utility question. Nevertheless, the database should be seen as the intermediator between the hardware and the website, e.g. using CouchDB to store the information.

The file storage capability may present the possibility of placing all the generated reports by the user in a separate system, specifically designed for data storage. As with the database, a user access mechanism may be used to maintain data integrity between different users and to keep data separated. Also, this storage can be migrated to a different location if needed.

Furthermore, the website is the main interface between the user and the rest of the system. As an example, using industry strength encryption to ensure that the transferred data is safe, the user can easily login from any location and have immediate access to the running experiments. Moreover, another example is to use Ruby on Rails, rendering the software to be easily migrated to a different location. Furthermore, the website suitably contains an administrator interface, allowing the service provider to properly configure and calibrate each system, assign a specific instrument to a specific user or reset passwords depending on the needs.

An Application Example

Below follows an example of an application for the system setup according to the present invention. In this study, the impact of two different loading rates of soy milk into four 2 L reactors was investigated. The test was carried out with manually feeding for 18 days at 37° C. For a normal procedure, 18 days is a too short time to perform a continuous fermentation process, however, an indication of the process performance can be achieved earlier, and the purpose of this test was mainly to verify the functionality and highlight an example of how the system setup can be utilised for anaerobic fermentation tests in continuous procedures.

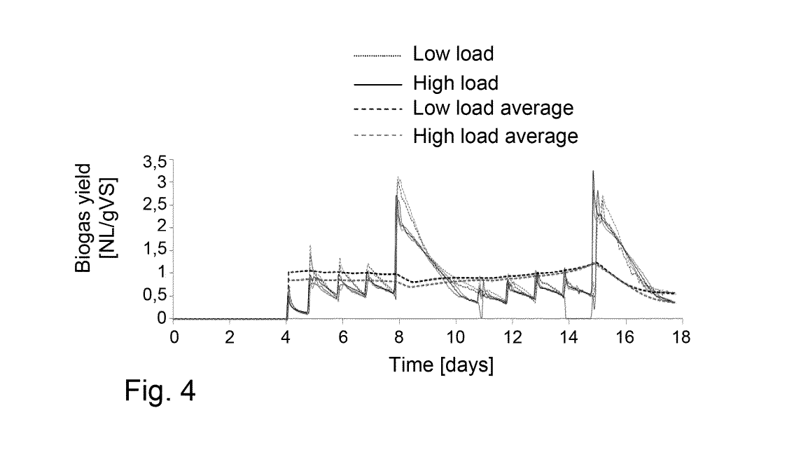

The results of the test are given in FIGS. 2-4 and are taken directly from a generated MS Excel report with the exception of minor manual input for calculation of average values. In FIG. 2, the organic loading rate is plotted. Here a typical trend can be seen with a higher value at weekdays and lower over the weekend when no feedings were made. The reason the lines are not completely straight is because each feeding is not made at exactly the same time which gives small variations in the reported values.

In FIG. 3, the biogas productivity of the four different reactors is plotted. No substrate was added into the reactors during the first four days; as seen, all the reactors have a similar declining profile in the biogas production during that period. After the feeding was started at day four, the two loading regimes start to differentiate. For the following days there is clear separation of gas production with a good reproducibility within the replicates. From day eight, the reactors are not fed for two days, therefore a severe declining trend can be observed again in biogas production. The fact that both loading regimes end up at a similar level at the end of this starvation period shows that most of the added easily biodegradable substrate has been consumed. The second week of the experiment has the same trend as week one, showing that the system is most probably at balance.

In FIG. 4, the biogas yield has been plotted. Since this is a direct correlation between the organic loading and gas productivity, a similar trend of the two parameters can be observed. The high peaks at days 8 and 15 are due to the lower organic loading at this time. In general, the test showed that the system could be used with minimal effort in both the operation and data acquisition and interpretation.

### CONCLUSIONS

The present invention provides a system setup with a cloud based platform, inter alia for anaerobic continuous fermentation tests and even full-scale process, which allows users to easily set-up, operate and follow-up experiments and process operations. By having the capability of utilising cloud computing it is possible to monitor and control the experiment from any location with internet access. Moreover, the system setup may guarantee that there is no limitation in data storage and computing capacity. The system is specially designed for operating laboratory-scale anaerobic digestion processes in order to simulate full-scale operation in continuous feeding/discharging mode. The system may calculate all the key process parameters in real-time and present these in a suitable fashion.

Some of the advantages with the system setup according to the present invention are:
No risk of data oversaturation. Since the majority of the experiments conducted in a continuous mode will be dealing with a large amount of data, it is of value to shift this from the physical system to another location. This allows the physical system to remain fairly simple without special need of large computational power or storage capacity.
Easy set-up of the system. Having the system automatically connected to a cloud setup also alleviates some of the internet configuration problems that can occur when dealing with localised setups. Data access can be achieved without the user having to configure local equipment for the intranet they are located at. No port forwarding or similar steps are needed as the system uploads all the data automatically at a port that is normally open for external communication if the connection originates on the internal network. When accessing the data, the connection will originate from the internal network on a port that is normally open and the connection will be made to the server hosting the website software. At no point there must be a need for a connection to originate from outside of the intranet.
Easy updating and maintenance. Hosting the user interface and the calculations on an external server under direct control of the service provider also makes updating the software with more features or fixing bugs much easier. There is much less of a need for users to manually patch the physical machine. Most solutions will involve upgrading the software running on the server and the advantages will be made immediately available to everyone. The file server also provides a very convenient solution for users to store their generated reports, which may be accessed at any time from any internet connected location.

To summarize, the present invention provides a system setup to use as a new process simulation tool designed e.g. for laboratory-scale experiments or process monitoring tool for full-scale operation. The flexible setup may have a user-friendly interface hosted in a cloud environment, an instrument platform that is able to perform continuous fermentation tests in a very accurate and reproducible manner with minimal work load for both operation follow up and data interpretation. Hosting the setup in a cloud environment offers great benefits in terms of unmatched accessibility, reliability and flexibility which are ideal for a data intensive continuous anaerobic fermentation test or operation. Furthermore, the system may offer high precision gas production monitoring that e.g. may rely on a flow meter array for ultra low gas flows using the principle of liquid displacement and buoyancy.

1. A system setup for monitoring and/or controlling one or multiple fermentation processes, said system setup comprising
at least one fermentation unit;
a data acquisition unit; and
a cloud computing unit having a database, a file storage capability, a data calculation capability and a user interface capability;
wherein the at least one fermentation unit is connected to the acquisition unit which in turn is connected to the cloud computing unit so that on-line, real-time data on the one or multiple fermentation processes may be transferred from the at least one fermentation unit via the data acquisition unit to the cloud computing unit to be interpreted and displayed for a user being on-line, and wherein the system set-up enables measuring in the at least one fermentation unit and/or the data acquisition unit of the on-line, real-time data on the one or multiple fermentation processes,
wherein the system setup also comprises
one or multiple laboratory simulation platform(s) and/or full-scale process(es) comprising said at least one fermentation unit, being in data connection with the cloud computing unit;
and wherein the data acquisition unit is connected to the cloud computing unit so that all data acquisition, data interpretation and data storage is performed centralized on the cloud computing unit.

2. System set-up according to claim 1, wherein all data acquisition, data interpretation and data storage being performed centralized is performed in a standardized manner.

3. System set-up according to claim 2, wherein the standardized manner implies data interpretation that allows for comparison and information sharing of the relevant data among users within a defined user group/community.

4. System set-up according to claim 2, wherein the standardized manner implies that established well-defined standards and protocols are used for relevant data and data presentation.

5. System set-up according to claim 2, wherein the standardized manner implies the use of a pre-defined format for data storage and data presentation.

6. System set-up according to claim 1, wherein the system set-up comprises one or multiple laboratory simulation platform(s).

7. System set-up according to claim 1, wherein the one or multiple fermentation processes are anaerobic or aerobic fermentation processes.

8. System set-up according to claim 1, wherein the one or multiple fermentation processes are one or multiple biogas producing fermentation processes.

9. System set-up according to claim 1, wherein the system set-up also includes one or several measuring devices and/or sensors.

10. System set-up according to claim 9, wherein one or several measuring devices and/or sensors are positioned in the data acquisition unit.

11. System set-up according to claim 8, wherein the system set-up includes at least one gas flow measuring device.

12. System set-up according to claim 8, wherein the system set-up also includes sensor(s) measuring pH, temperature, pressure, gas composition, ORP (oxygen redox potential), alkalinity, (dissolved) hydrogen or (dissolved) oxygen, VFA (volatile fatty acid), biodegradable organic matter, or any fermentation metabolites as key process parameters.

13. System set-up according to claim 8, wherein the system set-up also includes sensor(s) measuring pH, gas composition, (dissolved) hydrogen, or temperature, or a combination thereof.

14. System set-up according to claim 1, wherein the data acquisition unit holds both on-line, real-time data on the one or multiple fermentation processes and also user identity information to transfer data to a correct user account in the cloud computing unit.

15. Use of a system according to claim 1, for monitoring and/or controlling one or multiple fermentation processes.

16. Use according to claim 15, wherein said one or multiple fermentation processes are biogas producing fermentation processes.

17. Use according to claim 15, wherein at least one biogas flow is measured and is the on-line, real-time data on the one or multiple fermentation processes.

18. Use according to claim 17, wherein the at least one biogas flow is compensated with reference to temperature and pressure, which parameters are also measured.

19. Use according to claim 15, wherein monitoring and/or controlling is performed continuously on one or multiple continuous fermentation processes.
